(19) United States
(12) Patent Application Publication
(10) Pub. No.: US 2024/0280840 A1
(43) Pub. Date: Aug. 22, 2024

(54) ANTI-CONDENSATION PROTECTIVE EYEWEAR

(71) Applicant: GENTEX CORPORATION, SIMPSON, PA (US)

(72) Inventors: TAYLOR WORTHINGTON, DURYEA, PA (US); HAIPING YU, SIMPSON, PA (US)

(73) Assignee: GENTEX CORPORATION, SIMPSON, PA (US)

(21) Appl. No.: 18/571,638

(22) PCT Filed: Jul. 6, 2022
(86) PCT No.: PCT/US2022/036182

§ 371 Date: Dec. 18, 2023

Related U.S. Application Data
(60) Provisional application No. 63/219,130, filed on Jul. 7, 2021.

(51) Int. Cl. G02C 11/08; A61F 9/02; G02C 7/10

(52) U.S. Cl. CPC G02C 11/08; G02C 7/102; A61F 9/029

(57) ABSTRACT

An anti-condensation protective eyewear includes a lens having an outer periphery, an inner surface, and an outer surface. There is a sidewall structure coupled to the periphery of the lens and having an inner surface and an outer surface. There are one or more thermoelectric cooling modules coupled to the sidewall structure. Each of the thermoelectric cooling modules includes a cooling surface disposed on a first side, and a heating surface disposed on a second side, opposite the first side. The inner surface of the sidewall structure and the inner surface of the lens form an interior chamber, and the cooling surface of each of the one or more thermoelectric cooling modules is disposed within the interior chamber.

### CROSS-REFERENCE TO RELATED APPLICATIONS

This application claims the benefit of U.S. Provisional Patent Application No. 63/219,130 filed Jul. 7, 2021 entitled “Anti-Condensation Protective Eyewear”, which is incorporated by reference herein in its entirety.

### TECHNICAL FIELD

The present invention generally relates to protective eyewear and, more particularly, to protective eyewear having one or more thermoelectric cooling modules coupled thereto to prevent condensation build up on the lens of the protective eyewear.

### SUMMARY

In one embodiment, there is an anti-condensation protective eyewear including a lens having an outer periphery, an inner surface and an outer surface. The anti-condensation eyewear further includes a sidewall structure coupled to the periphery of the lens, the sidewall structure having an inner surface and an outer surface and one or more thermoelectric cooling modules coupled to the sidewall structure. Each thermoelectric cooling module of the one or more thermoelectric cooling modules includes a cooling surface disposed on a first side of the thermoelectric cooling module, and a heating surface disposed on a second side, opposite the first side, of the thermoelectric cooling module. The inner surface of the sidewall structure and the inner surface of the lens form an interior chamber and the cooling surface of each of the one or more thermoelectric cooling modules is disposed within the interior chamber.

In some embodiments, the sidewall structure includes a top surface, a bottom surface, a first side surface, and a second side surface, and the one or more thermoelectric cooling modules are coupled to the top surface. In some embodiments, the one or more thermoelectric cooling modules are coupled to at least one of the top surface and the first side surface and the second side surface. In some embodiments, the top surface, bottom surface, first side surface, and second side surface are integrally formed. In some embodiments, the one or more thermoelectric cooling modules includes three or more thermoelectric cooling modules. In some embodiments, the one or more thermoelectric cooling modules includes four thermoelectric cooling modules.

In some embodiments, the one or more thermoelectric cooling modules are configured to be electrically coupled to a mounting rail such that the one or more thermoelectric cooling modules receive power from the mounting rail. In some embodiments, the anti-condensation protective eyewear further includes a thermally conductive element coupled to the cooling surface and disposed within the interior chamber. In some embodiments, the sidewall structure is at least partially comprised of a flexible material. In some embodiments, the sidewall structure is comprised of silicon. In some embodiments, the anti-condensation protective eyewear further includes a heat sink coupled to the heating surface of each of the one or more thermoelectric cooling modules. In some embodiments, the heating surface of each of the one or more thermoelectric cooling modules is coupled to the outer surface of the sidewall structure and is exterior to the interior chamber.

In some embodiments, the one or more thermoelectric cooling modules are in electrical communication with a power source coupled to a helmet. In some embodiments, the lens is comprised of a ballistic grade material. In some embodiments, the lens includes one or more laser absorptive dyes, laser reflective coatings or a combination thereof. In some embodiments, the lens includes a hydrophobic coating. In some embodiments, the lens includes one or more variable light transmissive materials including at least one of a photochromic material, an electrochromic material, and liquid crystal technology. In some embodiments, the anti-condensation protective eyewear further includes a display device configured to project an image to a wearer's eye, the display device including one or more waveguides.

In some embodiments, the anti-condensation protective eyewear further includes at least one temperature sensor coupled to the inner surface of the sidewall structure, the inner surface of the lens or a combination of both, the at least one temperature sensor configured to generate a signal including an indication of at least one of a temperature inside the interior chamber and a temperature of the inner surface of the lens and a controller in communication with the at least one temperature sensor and coupled to the one or more thermoelectric cooling modules, the controller configured to selectively provide power to the one or more thermoelectric cooling modules in response receiving the signal from the at least one temperature sensor.

In some embodiments, the controller is configured to provide power to the one or more thermoelectric cooling modules in response to the signal indicating that the temperature inside the interior chamber or the temperature of the inner surface of the lens is above a predetermined threshold. In some embodiments, the controller is configured to cease providing power to the one or more thermoelectric cooling modules in response to the signal indicating that the temperature inside the interior chamber or the temperature of the inner surface of the lens is below a predetermined lower threshold. In some embodiments, the sidewall structure is a flexible gasket coupled to the periphery of the lens to form a continuous seal between the lens and flexible gasket.

In some embodiments, the one or more thermoelectric cooling modules extend through the sidewall structure. In some embodiments, the lens is a dual lens structure including an inner lens and an outer lens spaced from the inner lens, the space between the inner lens and outer lens defining a lens gap, and the heating surface of the one or more thermoelectric cooling modules is thermally connected to the lens gap. In some embodiments, heat generated by the heating surface of at least one of the one or more thermoelectric cooling modules is configured to be transferred to a location or device on or proximate a user wearing the anti-condensation protective eyewear.

In another embodiment there is an anti-condensation protective eyewear including a lens having an outer periphery, an inner surface, and an outer surface. There is a sidewall structure coupled to the periphery of the lens, the sidewall structure having an inner surface and an outer surface, a top surface, a bottom surface, a first side surface, and a second side surface, the inner surface of the sidewall structure and the inner surface of the lens defining an interior chamber. There are four or more thermoelectric cooling modules coupled to the sidewall structure. Each thermoelectric cooling module of the four or more thermoelectric cooling modules includes a cooling surface disposed on a first side of the thermoelectric cooling module, a heating surface disposed on a second side, opposite the first side, of the thermoelectric cooling module, a thermally conductive element coupled to the cooling surface, and a heat sink coupled to the heating surface. The four or more thermoelectric cooling modules include a first thermoelectric cooling module and a second thermoelectric cooling module each coupled to the top surface of the sidewall structure, a third thermoelectric cooling module coupled to the first side surface of the sidewall structure, and a fourth thermoelectric cooling module coupled to the second side surface of the sidewall structure. Each thermoelectric cooling module of the four or more thermoelectric cooling modules extend through the sidewall structure such that the cooling surface of each of the four or more thermoelectric cooling modules is disposed within the interior chamber.

### BRIEF DESCRIPTION OF THE SEVERAL VIEWS OF THE DRAWINGS

The following detailed description of embodiments of the anti-condensation protective eyewear, will be better understood when read in conjunction with the appended drawings of exemplary embodiments. It should be understood, however, that the invention is not limited to the precise arrangements and instrumentalities shown.

In the drawings:

FIG. 1 is a perspective view of an anti-condensation protective eyewear in accordance with an exemplary embodiment of the present disclosure;

FIG. 2 is a schematic cross-sectional view of a thermoelectric cooling module in accordance with an exemplary embodiment of the present disclosure;

FIG. 3 is a perspective view of an anti-condensation protective eyewear in accordance with another embodiment of the present disclosure;

FIG. 4 is a perspective view of an anti-condensation protective eyewear in accordance with another embodiment of the present disclosure;

FIG. 5 is a perspective view of the anti-condensation protective eyewear of FIG. 1 and a helmet system for coupling to the anti-condensation protective eyewear;

FIG. 6A is a side perspective view of an anti-condensation protective eyewear in accordance with another embodiment of the present disclosure;

FIG. 6B is a side perspective view of an anti-condensation protective eyewear in accordance with another embodiment of the present disclosure;

FIG. 7A is a cross-sectional perspective view of a dual lens structure in accordance with another embodiment of the present disclosure; and

FIG. 7B is a magnified cross-sectional perspective view of the dual lens structure of FIG. 7A.

### DETAILED DESCRIPTION

In various operating environments, sealed or substantially sealed protective eyewear, face or respiratory systems are often required for protection. In hazardous operating environments, fully sealed devices including protective eyewear (e.g., goggles), face masks, visors, helmets, and/or respiratory systems are used to provide protection from hazardous operating conditions/environments. Such protective eyewear is often prone to the buildup of condensation on the lens which may impair the vision of the wearer of the protective eyewear. In certain operating environments, it is not possible or practical to remove the protective eyewear to clear the condensation or “fog” from the lens. In such instances, condensation build up on the interior of the lens may be dangerous as the wearer operates within the hazardous environment. Furthermore, in sealed protective eyewear, the user's face, particularly the forehead, often transfers heat via thermal radiation to the air cavity formed between the interior of the lens and the user's face. During any physical exertion this heating may be expedited, and water may more quickly condensate on the interior surface of the lens. This may be caused in part by the increase in temperature of the air cavity and in part by perspiration on the user's face due to physical exertion and the increasing temperature of the air cavity. Because said protective eyewear is often sealed or at least partially sealed, the condensation may become more severe with time, further reducing the wearer's visibility.

Therefore, the present disclosure includes anti-condensation protective eyewear configured to cool air within the cavity/chamber formed between the interior of the lens and the wearer's face. Cooling the air within the cavity may prevent, or at least reduce, condensation from building up on the interior of the lens, allowing the visibility of the wearer to remain unobstructed due to said condensation build up. Additionally, cooling the air within the cavity may cool the portion of the wearer's face disposed opposite the lens (e.g., the wearer's forehead) causing a reduction in perspiration on the wearer's forehead and improving comfortability of the protective eyewear.

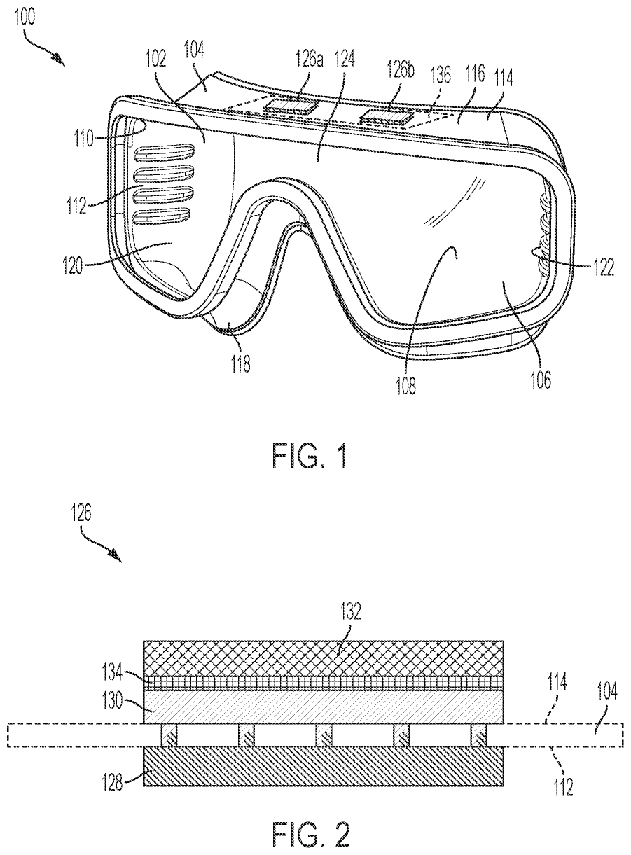

Referring to the drawings in detail, wherein like reference numerals indicate like elements throughout, there is shown in FIGS. 1-5 anti-condensation protective eyewear, generally designated 100, in accordance with an exemplary embodiment of the present invention.

Referring to FIG. 1, there is shown anti-condensation protective eyewear 100, also referred to as eyewear 100. The eyewear 100 may include a lens 102 and a sidewall structure 104 coupled to the lens 102. In some embodiments, the eyewear 100 is a goggle. In other embodiments, the eyewear may be glasses, a visor, a mask (e.g., gas mask), a face shield, or a fully enclosed head protection system. The lens may have an outer surface 106 and an inner surface 108 disposed opposite the outer surface 106. The lens 102 may be sized to extend across a portion of a wearer's face. In some embodiments, the lens 102 is sized to extend across the wearer's eyes and forehead. In some embodiments, the lens 102 may be comprised of a transparent material (e.g., plastic or glass). In some embodiments, the lens 102 may be comprised of a transparent material which is rigid. In other embodiments, the lens 102 may be comprised of a transparent material which is at least partially flexible. In some embodiments, the lens 102 may be comprised of a polycarbonate material. In some embodiments, the lens 102 may be double paned having two lenses spaced from one another to form a gap between the panes. In some embodiments, the gap may form a vacuum or be filled with air, argon, or another inert gas to function as a heat insulation layer which may be beneficial in low temperature operating environments.

The sidewall structure 104 may be coupled to an outer periphery 110 of the lens 102. The sidewall structure 104 may be configured to form a seal between the lens 102 and the portion of the user's face interior to the sidewall structure 104 when worn by the user. The sidewall structure 104 may provide additional protection to a user wearing the eyewear 100 (e.g., such as a component of an eye protection system for a helmet), and/or space the lens 102 from a user's face. The sidewall structure 104 may have an inner surface 112 and an outer surface 114 disposed opposite the inner surface 112. In some embodiments, the sidewall structure 104 may extend rearwardly from the outer periphery 110 of the lens 102. In some embodiments, the sidewall structure 104 may include a top surface 116, a bottom surface 118, a first side surface 120, and a second side surface 122. In some embodiments, the top surface 116, bottom surface 118, first side surface 120, and second side surface 122 are integrally formed. In some embodiments, the sidewall structure 104 may be at least partially comprised of a flexible material to allow the sidewall structure to conform to the shape of a wearer's face. In some embodiments, the sidewall structure 104 may be comprised of silicon. In some embodiments, the sidewall structure 104 is a flexible gasket coupled to the outer periphery 110 of lens 102. In some embodiments, the flexible gasket may form a continuous seal between the lens 102 and flexible gasket.

In some embodiments, the inner surface 112 of the sidewall structure 104 and the inner surface 108 of the lens 102 may define an interior chamber 124. The interior chamber 124 may define the area between the lens 102, the sidewall structure 104, and a user's face when wearing the eyewear 100. When in use, air within the interior chamber 124 of the eyewear 100 may be sealed, at least mostly sealed, within the interior chamber 124 by the seal formed between the wearer's face, the inner surface 112 of the sidewall structure and the inner surface 108 of lens 102. The moisture within the air contained within the interior chamber 124 may be a source of condensation when the surface temperature of the inner surface 108 of lens 102 is at or below the dew point temperature of air within the interior chamber 124. The dew point temperature may be dependent upon the temperature of the air within the interior chamber 124 such that the lower the temperature of the air within the interior chamber 124, the lower the dew point temperature is, and vice versa. The dew point temperature may also depend on the humidity of the air within the interior chamber 124. In some embodiments, condensation build up on the inner surface 108 of lens 102 may be reduced and/or prevented by reducing the temperature of the air contained within the interior chamber 124 such that the temperature of the inner surface 108 of the lens 102 is greater than the dew point temperature.

By reducing the temperature of the atmosphere within the interior chamber 124, the dew point temperature may be reduced creating a temperature gradient making the inner surface 108 of the lens 102 a higher temperature than the atmosphere within the interior chamber 124, thereby causing any condensation build up on the inner surface of the lens 102 to evaporate back into the atmosphere of the interior chamber 124. Reducing the temperature of the atmosphere within the interior chamber 124 also reduces the humidity of said atmosphere further preventing the inner surface 108 of the lens 102 from fogging. Reducing the temperature of the atmosphere within the interior chamber 124 may also provide a cooling sensation to the user wearing the protective eyewear 100 reducing and/or preventing perspiration from the user, thereby further reducing moisture generation within the interior chamber 124.

In some embodiments, the protective eyewear 100 may include one or more thermoelectric cooling (TEC) modules 126 configured to reduce the temperature of the atmosphere within the interior chamber 124. Referring to FIG. 2, in some embodiments, each TEC module 126 may be configured to provide heat transfer and dissipation capabilities to the eyewear 100. A TEC module 126, also referred to as a thermoelectric cooler or Peltier cooler, may be a semiconductor-based electronic component configured to transfer heat from one side (e.g., a first side) of the TEC module 126 to the other side (e.g., the second side) of the TEC module 126 in response to receiving an electrical signal (e.g., a voltage). Each TEC module 126 may include a cooling surface 128 disposed on a first side of the TEC module 126 and a heating surface 130 disposed on a second side, opposite the first side, of the TEC module 126. In some embodiments, the cooling surface 128 may be coupled to the inner surface 112 of sidewall structure 104 and the heating surface 130 may be coupled to the outer surface 114 of sidewall structure 104 such that the TEC module 126, when in use, transfers heat from the interior chamber 124 to the external atmosphere.

In some embodiments, there may be a heat sink 132 coupled to the heating surface 130 to dissipate heat from the heating surface 130 to the environment external to the interior chamber 124. In some embodiments, the heat sink 132 may include structures and/or materials configured to dissipate heat from the heating surface 130. For example, the heat sink 132 may include a lattice and/or aluminum honeycomb structure, a metal foam, and/or micro fans to dissipate heat from the heating surface 130. In some embodiments, there may be an adhesive heat conductor 134 disposed between the heating surface 130 and the heat sink 132 coupling the heat sink 132 to the heating surface 130. Each TEC module may have a minimum thickness on the order of ˜2 millimeters and may be manufactured in an array of shapes and sizes. In some embodiments, each TEC module may have a thickness in a range of about 1 millimeter to about 10 millimeters.

Referring to FIG. 1, in some embodiments, the one or more TEC modules 126 may be coupled to the sidewall structure 104. In some embodiments, each of the one or more TEC modules 126 is coupled to the sidewall structure 104 such that the cooling surface 128 is disposed within the interior chamber 124. In some embodiments, there may be a first TEC module 126a and a second TEC module 126b coupled to the top surface 116 of the sidewall structure 104. In some embodiments, the first TEC module 126a and second TEC module 126b may extend through the sidewall structure 104 such that the heating surface 130 of each TEC module 126a, 126b is exterior to the interior chamber 124 and the cooling surface 128 is disposed within the interior chamber 124. In some embodiments, the TEC modules 126a, 126b extend through sidewall structure 104 and form a seal with the sidewall structure such that the atmosphere external to the interior chamber 124 is not able to enter the interior chamber 124 through the area in which the TEC modules 126a, 126b extend through the sidewall structure 104. In this manner, the TEC modules 126a, 126b may transfer heat from the interior chamber 124 to the external atmosphere while remaining sealed.

In some embodiments, there is a thermally conductive element 136 coupled to the cooling surface 128 of each TEC module 126a, 126b and configured to improve the heat transfer capabilities of the TEC modules 126a, 126b. The thermally conductive element 136 may be a thermally conductive sheet, vapor chamber or heat pipe, or a thermally conductive material. The thermally conductive element 136 may be disposed within the interior chamber 124 and have a surface area which is greater than the surface area of the cooling surface 128 of each TEC module 126a, 126b to increase the overall cooling area within the interior chamber 124. In FIG. 1, a single thermally conductive element 136 is coupled to the cooling surface 128 of both TEC modules 126a, and 126b, however there may be a separate and distinct thermally conductive element 136 coupled to the cooling surface 128 of each TEC module 126a, 126b.

In some embodiments, the thermally conductive element 136 may be comprised of a thermally conductive material. In some embodiments, the thermally conductive element 136 may be one or more heat pipes or vapor chambers contacting the cooling surface 128 of each TEC module 126a, 126b. A heat pipe may refer to a micro-fluidic device configured to form a thermal cycle using a minute amount of working fluid driven by capillary action which undergoes phase transformations to effect heat transfer. A vapor chamber may refer to a thinner, or flatter, version of a conventional heat pipe. By providing a thermally conductive element 136, the cooling and humidity reducing capabilities of each TEC module 126a, and 126b may be improved.

In some embodiments, the heating surface 130 of each TEC module 126a, and 126b may be coupled to the outer surface 114 of the sidewall structure 104 such that the heating surface 130 is exterior to the interior chamber 124. Dissipation of heat from the heating surface 130 of each TEC module 126a, 126b may be improved (e.g., improved efficiency, improved rate of heat transfer) by providing additional structures and/or devices to the heating surface 130 of each TEC module 126a, 126b. Although not depicted in FIG. 1, the heat sink 132 described with reference to FIG. 2, may be coupled to the heating surface 130 of each TEC module 126a, 126b to improve efficiency of the heat transfer from the heating surface 130 to the ambient environment external to the interior chamber 124. The heat sink 132 may be a passive finned heat sink. In some embodiments, one or more TEC modules 126 may be stacked to improve heat dissipation from the heating surface 130 of the TEC module 126 directly coupled to the sidewall structure 104. For example, additional TEC modules 126 may be coupled to each of TEC modules 126a and 126b such that the cooling surface 128 of the additional TEC modules 126 contacts the heating surface 130 of TEC modules 126a, 126b. In other embodiments, active micro-fans and/or vapor chambers may be coupled to the heating surface 130 of TEC modules 126a, 126b to improve heat dissipation from the heating surfaces 130.

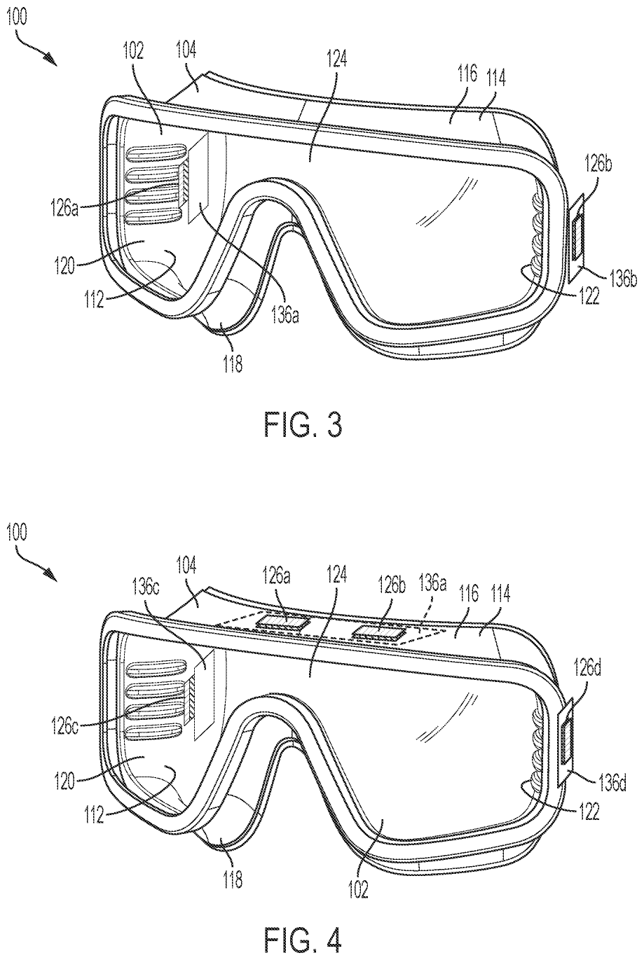

In FIG. 1, the TEC modules 126a, and 126b are arranged along the top surface 116 of the sidewall structure 104, however the protective eyewear 100 may have TEC modules 126 arranged along a combination of surfaces of the sidewall structure. For example, referring to FIG. 3, in some embodiments, TEC module 126a may be coupled to the first side surface 120, and TEC module 126b may be coupled to second side surface 122 of the sidewall structure 104. The TEC modules 126a, 126b may each extend through the first side surface 120 and second side surface 122, respectively, of the sidewall structure 104 such that the cooling surface 128 is within the interior chamber 124 and the heating surface 130 is exterior to the interior chamber 124. In some embodiments, there may be thermally conductive elements 136a, 136b coupled to the cooling surface 128 of each TEC module 126a, and 126b respectively. The thermally conductive elements 136a, and 136b may each have a surface area which is greater than the surface area of the cooling surface 128 of the TEC modules 126a, and 126b respectively. Similar to the arrangement shown in FIG. 1, the thermally conductive elements 136a, 136b may each be positioned within the interior chamber 124. Although not shown in FIG. 3, a heat sink 132 may be coupled to the heating surface 130 of each TEC module 126a, 126b.

Referring to FIG. 4, in some embodiments, the protective eyewear 100 may include four or more TEC modules 126 arranged along one or more surface of the sidewall structure 104. In some embodiments, the protective eyewear 100 may include a first TEC module 126a, and a second TEC module 126b each coupled to the top surface 116 of the sidewall structure 104. The first and second TEC modules 126a, 126b may each extend through the top surface 116 of the sidewall structure 104 such that the cooling surface 128 of each TEC module 126a, 126b is disposed within the interior chamber 124. In some embodiments, the protective eyewear 100 may include a third TEC module 126c coupled to the first side surface 120 of the sidewall structure 104. In some embodiments, the protective eyewear 100 may include a fourth TEC module 126d coupled to the second side surface 122 of the sidewall structure 104. The third and fourth TEC modules 126c, 126d may each extend through the respective first and second side surfaces 120, 122 of the sidewall structure 104 such that the cooling surface 128 of the respective third and fourth TEC modules 126c, 126d are disposed within the interior chamber 124. The heating surfaces 130 of each TEC module 126a-126d may be coupled to the outer surface 114 of the sidewall structure such that said heating surfaces 130 are positioned exterior to the interior chamber 124. In some embodiments, one or more TEC modules 126 may be coupled to the bottom surface 118 of the sidewall structure 104 such that the cooling surface 128 of said TEC modules 126 are within the interior chamber 124.

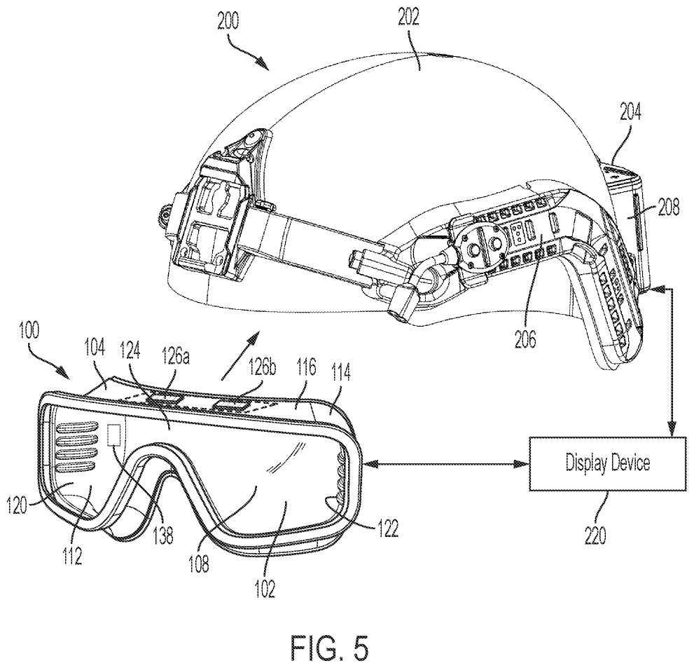

Referring to FIG. 5, the anti-condensation protective eyewear 100 may be configured to couple to a helmet system, generally designated 200, in accordance with an exemplary embodiment of the present disclosure. In some embodiments, the helmet system 200 may include a helmet shell 202, a power source 204, and a mounting rail 206 in electrical communication with the power source 204. In some embodiments, the power source 204 may be mounted to the helmet shell 202. In other embodiments, the power source 204 may be external to the helmet shell 202 and the eyewear 100. In some embodiments, the mounting rail 206 may include a plurality of electrical connections extending along an interior surface of the mounting rail electrically coupling the mounting rail 206 to the power source 204. In some embodiments, the eyewear 100 may be configured to receive power from the power source 204 when the eyewear 100 is coupled to the helmet system 200. In some embodiments, the eyewear 100 may be electrically coupled to the mounting rail 206.

The embodiment shown in FIG. 5 includes two TEC modules 126a, and 126b in the configuration shown in FIG. 1. It will be understood however, that any of the configurations shown in FIGS. 1, 3, and 4 may be coupled to the helmet system 200 such that the TEC modules 126 receive power from the power source 204. In some embodiments, the TEC modules 126 coupled to the sidewall structure 104 may receive power from power source 204 to effect heat transfer between the atmosphere within the interior chamber 124 and the atmosphere exterior to the interior chamber 124. For example, power may be transmitted from the power source 204, along the electrical connections within the mounting rail 206 to the TEC modules 126a, and 126b.

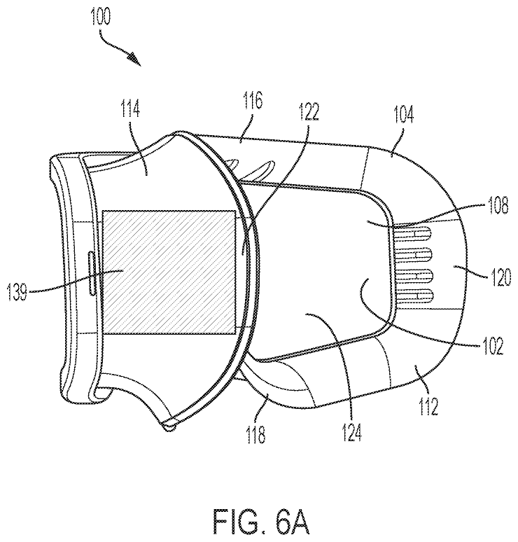

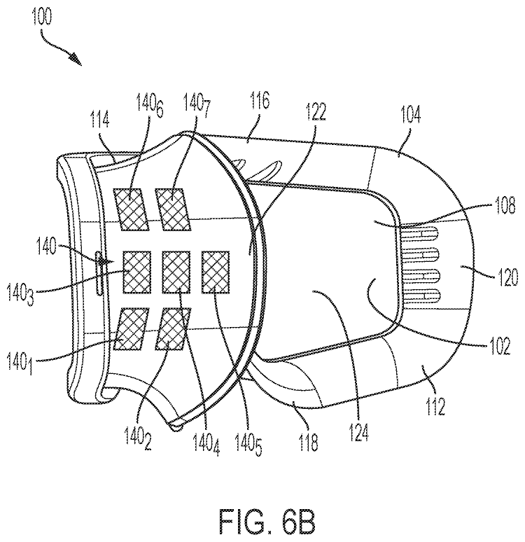

Referring to FIG. 6A, a TEC module 139 being generally larger in size than TEC modules 126, as described in FIGS. 1-5, may be coupled to the sidewall structure. The TEC module 139 may be generally the same as TEC modules 126, except that the TEC module 139 is larger in size to provide a greater cooling capacity when compared to the cooling capacity of a single TEC module 126. TEC module 139 may be coupled to sidewall structure 104 similar to TEC modules 126, as described above, such that a cooling surface of TEC module 139 is disposed within interior chamber 124 and a heating surface of TEC module 139 is exterior to the outer surface 114 of sidewall structure 104. The power source 204 may be configured to provide power to TEC module 139 in a similar manner as described above with reference to FIG. 5 and power source 204 providing power to TEC modules 126.

Referring to FIG. 6B, in some embodiments a TEC array 140 may be coupled to sidewall structure 104 instead of one or more of the arrangements of TEC modules 126 shown in FIGS. 1, and 3-5. For example, as shown in FIGS. 3-4, a single TEC module 126 is coupled to the first and second side surfaces 120, 122 and, as shown in FIGS. 1, 4, and 5, a one column by two row array of TEC modules is coupled to the top surface 116 of the sidewall structure 104. Any one of these arrangements may be replaced by a TEC array 140. In the embodiment shown in FIG. 6B, the TEC array 140 includes six TEC modules 140₁-140₄ and 140₆-140₇ arranged in two columns and three rows and another TEC module 1405 positioned in the middle row, for a total of 7 TEC modules 140₁-140₇. However, other arrangements may replace a single TEC module 126 or the array shown in FIGS. 1, 4 and 5. The TEC array 140 may be defined as two or more TEC modules distributed on one or more surfaces (e.g., top surface 116, bottom surface 118, first side surface 120, second side surface 122) of the sidewall structure 104. In some embodiments, each TEC module 140₁-140₇ included in the TEC array 140 is generally the same as TEC modules 126, as described in FIGS. 1-5, except that the TEC modules 140₁-140₇ are generally smaller in size than the TEC modules 126 shown in FIGS. 1-5. The TEC modules 140₁-140₇ may each be coupled to the sidewall structure 104 similar to TEC modules 126, as described above, such that a cooling surface of TEC modules 140₁-140₇ is disposed within interior chamber 124 and a heating surface of TEC modules 140₁-140₇ is exterior to the outer surface 114 of sidewall structure 104. The power source 204 may be configured to provide power to each TEC module 140₁-140₇ included in the TEC array individually or in group based on the application or to match the power capabilities of the power source 204. In the embodiment shown in FIG. 6, the TEC array 140 includes seven TEC modules 140₁-140₇. However, the TEC array 140 may include two, three, four, five, six, seven, or more than seven TEC modules. The TEC array 140, for example as shown in FIG. 6, may replace or accompany any existing singular TEC module configuration as shown in FIGS. 1-5. In some embodiments, the rows and columns of TEC modules 140₁-140₇ included in the TEC array 140 may be generally aligned. In other embodiments, the rows and columns of TEC modules 140₁-140₇ included in the TEC array 140 may not be generally aligned.

By providing a TEC array 140, as shown in FIG. 6, as opposed to a single TEC module, as shown in FIGS. 1-5, the flexibility in the mechanical integration of the TEC modules 140₁-140₇ may be increased. For example, the TEC modules 140₁-140₇ are arranged in the rows and columns as shown in FIG. 6B to allow the TEC modules 140₁-140₇ to conform to the shape of the sidewall structure 104. Additionally, in operating conditions where higher cooling capacities of the TEC modules are required to accomplish the anti-fog functionality the overall size of the TEC modules may need to be increased. However, TEC modules are typically solid nonflexible devices, and as the size of said TEC modules is increased (e.g., TEC module 139 shown in FIG. 6A) to provide higher cooling capacities, the larger TEC modules may become increasingly difficult to integrate with the curved and/or uneven non-flat surfaces of the sidewall structure 104.

Additionally, the surfaces of the sidewall structure 104 may be generally flexible such that the surfaces of the sidewall structure 104 may be deformed or bent during use or handling of the eyewear 100. For example, the sidewall structure 104 may be comprised of a generally flexible material and one or more surfaces of the sidewall structure 104 may be curved, such as the junction from top surface 116 to second side surface 122. Additionally, the surfaces of the sidewall structure 104 may be uneven and/or non-flat such as the top surface 116, bottom surface 118, and/or the first and second side surfaces 120 and 122. The TEC modules 140₁-140₇ included in the TEC array 140 may be positioned and oriented along any one of said surfaces 116, 118, 120, 122 to have an improved fit to the curved and/or uneven non-flat shape of said surfaces 116, 118, 120, 122, as shown in FIG. 6, when compared to a single larger TEC module. To this effect, by providing a TEC array 140 as opposed to a single larger TEC module, generally the same cooling capacity of a singular large TEC module may be achieved while also allowing the TEC modules 140₁-140₇ included in the TEC array 140 to better integrate with the curved, and/or uneven non-flat surfaces of the sidewall structure 104 and without negatively impacting the flexibility of the sidewall structure 104.

In some embodiments, variable cooling capacities associated with the activation of different amounts of TEC modules 140₁-140₇ may be required in different applications and/or operating conditions. For example, in some operating conditions only a small cooling capacity, associated with the activation of fewer than all the TEC modules 140₁-140₇ (e.g., half of the TEC modules 140₁-140₇), may be required to accomplish the anti-fog functionality. Because the TEC modules 140₁-140₇ of TEC array 140 may each be powered individually or in group, based on the application and/or operating conditions, only a subset of TEC modules 140₁-140₇ may be powered by power source 204 instead of the entire TEC array 140. As a result, the TEC array 140 may improve the efficiency of power usage and increase the amount of time (e.g., runtime) that the power source 204 may provide power to the TEC modules 140₁-140₇ of TEC array 140. Additionally, by selectively providing power to individual TEC modules 140₁-140₇, the energy conversion efficiency from electrical power stored in the power source 204 to the cooling capacity of the TEC array 140 may be increased, thereby optimizing the cooling capacity of the TEC array 140 regardless of the required amount of cooling (e.g., number of TEC modules 140₁-140₇ that are selectively powered). Thus, the TEC array 140 may allow for better power management by causing the individual TEC modules 140₁-140₇ to operate at a peak energy conversion efficiency, which also results in a longer runtime.

Referring back to FIG. 5, in some embodiments, the power source 204 is enclosed within a housing 208 coupled to the helmet shell 202. In some embodiments there is a controller in communication with the power source 204 and enclosed within housing 208. The controller may include a simple logic circuit and/or a micro-processor configured to control the power transmitted from the power source 204 to the TEC modules 126a, 126b coupled to the eyewear 100. In some embodiments, the controller may be configured to selectively provide power to the TEC modules 126a, 126b coupled to the eyewear 100 in response to the temperature of the atmosphere within the interior chamber 124. In some embodiments, there may be one or more temperature sensors 138 coupled to the eyewear and configured to generate a signal including an indication of the temperature inside the interior chamber 124. In some embodiments, the temperature sensor 138 may be coupled to the inner surface 112 of the sidewall structure 104. In the embodiment shown in FIG. 5, the temperature sensor 138 is coupled to the inner surface 112 of the first side surface of sidewall structure 104 however, the temperature sensor may be coupled to any portion of the sidewall structure 104. In some embodiments, one or more temperature sensors 138 may be arranged at various locations around the inner surface 112 to measure the temperature of the inner surface 108 of the lens 102.

In some embodiments, the temperature sensor 138 may be in communication with the controller and configured to transmit the signal, including the indication of the temperature, to the controller. The controller may be configured to receive the signal and selectively provide power to the TEC modules 126a, 126b based on the received signal from the temperature sensor. For example, if the signal received from the temperature sensor 138 indicates that the temperature inside the interior chamber 124 and/or the surface temperature of the inner surface 108 of the lens 102 is at or exceeds a predetermined upper threshold, the controller may transmit power from the power source 204 to one or more of TEC modules 126a, and 126b causing the TEC modules 126a and/or 126b to activate thus transferring heat from the interior chamber 124 to the external atmosphere and cooling the atmosphere within the interior chamber 124. If the signal received from the temperature sensor 138 indicates that the temperature inside the interior chamber is at or below a predetermined lower threshold, the controller may cease transmitting power from the power source 204 to one or more of the TEC modules 126a, and 126b causing the TEC modules 126a and/or 126b to cease transferring heat from the interior chamber 124 to the external atmosphere and allowing the temperature within the interior chamber 124 to increase.

By measuring and monitoring the temperature within the interior chamber 124, power may be more efficiently provided to the TEC modules 126 coupled to the sidewall structure 104 such that the humidity inside the interior chamber 124 is continually condensed and the inner surface 108 of lens 102 remains free of condensation. In some embodiments, the controller may provide power to one TEC module 126 separately from another TEC module 126 to improve power consumption.

In some embodiments, there may be one or more other temperature sensors exterior to the interior chamber 124 for measuring the temperature of the exterior atmosphere. In some embodiments, the one or more other temperature sensors may be in communication with the controller such that a temperature difference between the temperature of the external atmosphere and the atmosphere within the interior chamber 124 may be determined. In some embodiments, the controller may be configured to adjust the amount of power suppled to the TEC modules 126 coupled to the sidewall structure 104 based on the determined temperature differential.

In some embodiments, a material having absorbent properties (e.g., a sponge like material) may be disposed within the interior chamber 124 for collecting condensed water. In other embodiments, a desiccant material may be disposed within the interior chamber 124 for collecting condensed water. In some embodiments, the desiccant material may be a permanent desiccant material, a regeneratable desiccant material or a disposable desiccant material. The desiccant material may be any one of a silica gel, clay, zeolites, a metal organic framework, or a combination thereof. In some embodiments, the sponge like material and/or desiccant material may be used to absorb liquid water and reduce the humidity of the atmosphere within the interior chamber 124.

In some embodiments, a passive hydrophobic coating or a hydrophilic coating may be applied to the inner surface 108 of lens 102 to, in conjunction with any arrangement of TEC modules 126 discussed above, prevent condensation build up on the inner surface 108 of the lens 102 and/or to prevent initial condensation build up on the inner surface 108 of the lens 102 before the TEC modules 126 are fully active. In some embodiments, the inner surface 108 of lens 102 may be heated by one or more heating elements to prevent initial condensation build up and/or when the eyewear 100 is used in low temperature environments. In some embodiments, the one or more heating elements may be used in conjunction with any arrangement of the TEC modules 126 as discussed above. In some embodiments, the heating elements are in communication with the controller. In some embodiments, the controller is configured to selectively activate the heating elements to raise the surface temperature of the inner surface 108 of the lens 102. In other embodiments, the eyewear 100 may include a user operable input (e.g., a button) configured to activate the one or more heating elements to increase the surface temperature of the inner surface 108 of lens 102.

In some embodiments, the inner surface 108 of lens 102 may include an anti-reflection coating with a hydrophobic topcoat to prevent any water or sweat droplets from wetting the inner surface 108, which may obscure the user's vision. For example, during prolonged use of eyewear 100 an amount of sweat may be collected inside the interior chamber 124 of eyewear 100 and due to the sealed nature of the interior chamber 124, the sweat may not be expelled. To prevent the possibility of the sweat forming into large water droplets and the possibility of frost sticking to the inner surface 108, the hydrophobic coating is used. The hydrophobic coating may force larger water droplets, that require more energy to evaporate, to fall off the inner surface 108 of lens 102 and any small water residue remaining on the inner surface 108 may be evaporated according to the systems discussed above. In this manner, the power consumption of the eyewear 100 may be reduced, by not requiring additional power to evaporate large water droplets, thereby increasing runtime of the eyewear 100.

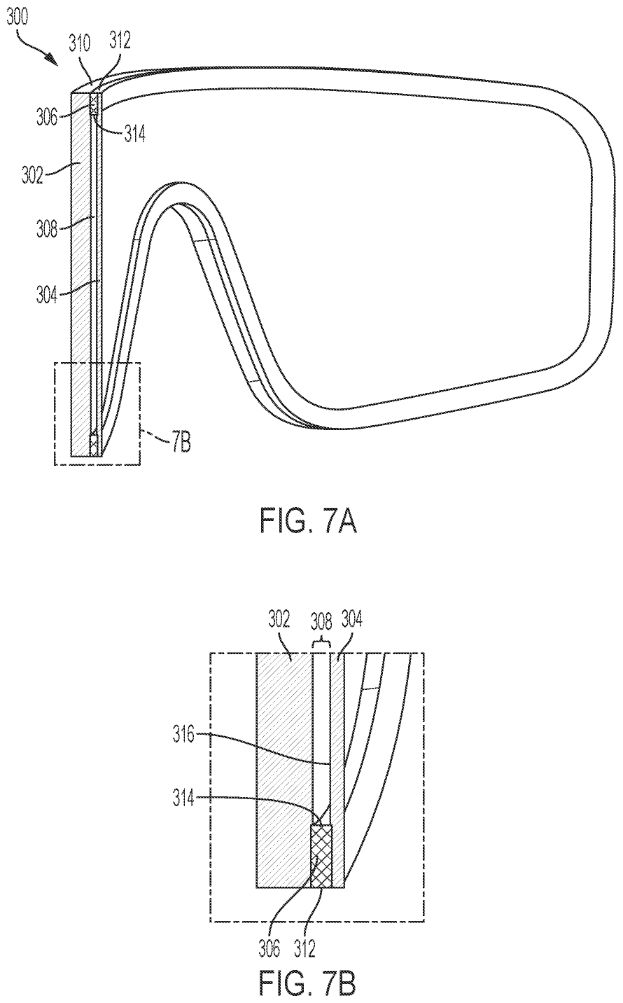

Referring to FIGS. 7A-7B, the eyewear 100 may include a dual lens structure, generally designated 300, as opposed to the lens 102 shown in FIGS. 1-6. Lens 102, as shown and described with reference to FIGS. 1-6, may be replaced with dual lens structure 300. The dual lens structure 300 may include an outer lens 302, an inner lens 304 spaced from the outer lens 302. When coupled to the sidewall structure 104 as shown in FIGS. 1-6, the inner lens 304 of dual lens structure 300 may define the interior chamber 124 similar to how the inner surface 108 of lens 102 and the sidewall structure 104 define the interior chamber 124. In some embodiments, there is a thermally conductive material 306 positioned in the space between the outer lens 302 and inner lens 304 proximate a periphery of the outer lens 302 and inner lens 304. In some embodiments, the thermally conductive material 306 is generally aligned with a periphery of the outer lens 302 and inner lens 304. An outer surface 312 of the thermally conductive material 306 may be positioned proximate and generally level with an outer surface 310 of outer lens 302. The thermally conductive material 306 may extend from the outer surface 312, inwardly along the space between the outer lens 302 and inner lens 304, to an inner surface 314.

In some embodiments, there may be a lens gap 308 defined by the space between the outer lens 302 and inner lens 304 and the inner surface 314 of the thermally conductive material 306. In some embodiments, the thermally conductive material 306 may form a seal between the outer lens 302 and inner lens 304 such that space within the lens gap 308 is sealed from the external environment. In this manner the lens gap 308 may provide an amount of thermal insulation between the outer lens 302 and inner lens 304. In some embodiments, the outer lens 302 and inner lens 304 have generally the same shape. In some embodiments, the thickness of the outer lens 302 may be greater than the thickness of the inner lens 304. In other embodiments, the thickness of the outer lens 302 may be generally the same or less than the thickness of the inner lens 304.

In the embodiments discussed above with reference to FIGS. 1-6, the cooling surface of the respective TEC modules 126, 140₁-140₇ is faces towards and is disposed within the interior chamber 124, and the corresponding heating surfaces are external to the interior chamber such that they face toward the ambient environment, or toward a heat sink coupled to said heating surface and dissipating the heat to ambient environment. In some embodiments, the heat energy from the heating surface of a respective TEC module 126, 140₁-140₇, as discussed above, may be recycled by thermally transferring the heat energy into the space within the lens gap 308 and/or to a surface 316 of inner lens 304 directly in contact with the air in the lens gap 308 (e.g., the convex surface 316 of the inner lens 304 that faces towards the lens gap 308). By transferring heat from a heating surface of one or more corresponding TEC modules 126, 140₁-140₇ to the lens gap 308 and/or surface 316 of inner lens 304, the temperature of surface 316 may be increased to a temperature greater than the dew point temperature, thereby preventing and/or eliminating condensation build up on inner lens 304.

By directly transferring heat energy from the heating surface of one or more corresponding TEC modules 126, 140₁-140₇ the temperature of surface 316 may be increased at a higher rate than when compared to heat transfer from the ambient environment. The higher heat transfer rate may allow the eyewear 100 to achieve a condensation free state more rapidly by increasing the difference in the temperature between the inner lens 304 and interior chamber 124 more rapidly. Furthermore, by recycling the heat energy from the heating surface of one or more corresponding TEC modules 126, 140₁-140₇, the power required to operate the TEC modules 126, 140₁-140₇ may be reduced thereby prolonging the runtime of power source 204 (as discussed with reference to FIG. 5). Inclusion of the dual lens structure 300 may also improve the operation of eyewear 100 in certain conditions by preventing the inner lens 304 from reaching a temperature below the dewpoint temperature, which may be possible in certain extreme cold operating conditions or environments.

In some embodiments the thermally conductive material 306 may include one or more portions (not shown) that directly contact the heating surface of a corresponding TEC module 126, 140₁-140₇ such that heat energy is transferred from said heating surface, along the one or more portions through the thermally conductive material 306 and into lens gap 308. In other embodiments, a heat pipe or vapor chamber may be used to transfer the heat energy to the lens gap 308. In some embodiments, a thermally conductive transparent coating such as transparent conductive oxides, ITO, metal meshes, or graphene composite coatings may be applied to the inner lens 304 and then thermally connected, via thermally conductive material 306 and/or heat pipes or vapor chambers, with the heating surface of a TEC module 126, 140₁-140₇, to provide a more uniform heating across surface 316 of inner lens 304.

In some embodiments, the heating surfaces of TEC array 140 may be connected to and transfer the heat to one or more locations and/or devices on or proximate the user such that the temperature of said location or device is raised. For example, in extreme cold weather environments the heat energy generated from the heating surface of the TEC 126, 140₁-140₇, may be transferred to an electro-optic device for quicker switching time, or to the power source 204 thus warming the power source 204 and improving performance thereof. In some embodiments, there may be one or more thermally conductive elements connected to one or more of the heating surfaces of the TEC array 140 and to one or more locations and/or devices on or proximate the user. In this manner, heat generated from the heating surface(s) may be transferred directly or indirectly to the user. In some embodiments, the TEC module(s) 126, as illustrated and described above with regards to FIGS. 1-5 may be configured to transfer heat generated at a respective heating surface 130 to one or more locations and/or devices on or proximate the user in generally the same manner.

In some embodiments, eyewear 100 may include light attenuating features such as variable light transmittance or light filtering features based on the photochromic, electrochromic or liquid crystal technologies. In such embodiments, the switch or transmittance change speed of said variable light transmittance or light filtering features may be negatively impacted by the temperature of the eyewear 100 or the ambient temperature. For example, cold temperatures may negatively impact the switch or transmittance change speed. By transferring heat energy into lens gap 308, as discussed above, the dual lens structure 300 may be capable of maintaining a temperature above ambient temperature in cold environments and thereby having a positive effect on operation of the light attenuating devices.

In some embodiments, the eyewear 100 may include a display device 220 configured to generate and/or project an image that is visible to the user either through the lens 102, reflected off lens 102 to the user's eye, or directly from a wave guide system residing between the user's eye and lens 102. For example, the display device 220 may include one or more waveguides for projecting an image to a wearer's eye. In some embodiments, the display device 220 may be contained within the lens 102 shown in FIG. 1 or outer lens 302 shown in FIG. 7. In some embodiments, the display device 220 may be a heads-up display (HUD). In some embodiments, the display device 220 may be electrically connected to power source 204 shown in FIG. 5 such that the display device 220 may receive power from the power source 204. In other embodiments, the display device 220 may receive power from a power source other than power source 204.

The anti-condensation protective eyewear and embodiments thereof shown and described above may be integrated with other components such as ballistic protection, laser eye protection, and variable light transmission systems. In some embodiments, the lens 102, 302, and/or 304 is comprised of a ballistic grade transparent material. Laser eye protection systems may include laser absorptive dyes, laser reflecting coatings or combinations thereof. Variable light transmission systems may include photochromic, electrochromic and liquid crystal technologies.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. The words “right”, “left”, “lower” and “upper” designate directions in the drawings to which reference is made. The words “inwardly” and “rearwardly” refer to directions toward and away from, respectively, the geometric center of the anti-condensation protective eyewear. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

1. An anti-condensation protective eyewear comprising:
a lens having an outer periphery, an inner surface, and an outer surface;
a sidewall structure coupled to the periphery of the lens, the sidewall structure having an inner surface and an outer surface; and
one or more thermoelectric cooling modules coupled to the sidewall structure, each thermoelectric cooling module of the one or more thermoelectric cooling modules comprising:
a cooling surface disposed on a first side of the thermoelectric cooling module; and
a heating surface disposed on a second side, opposite the first side, of the thermoelectric cooling module,
wherein the inner surface of the sidewall structure and the inner surface of the lens form an interior chamber, and
wherein the cooling surface of each of the one or more thermoelectric cooling modules is disposed within the interior chamber.

2. The anti-condensation protective eyewear of claim 1, wherein the sidewall structure includes a top surface, a bottom surface, a first side surface, and a second side surface, and
wherein the one or more thermoelectric cooling modules are coupled to the top surface.

3. The anti-condensation protective eyewear of claim 2, wherein the one or more thermoelectric cooling modules are coupled to at least one of the top surface and the first side surface and the second side surface.

4. The anti-condensation protective eyewear of claim 1, wherein the one or more thermoelectric cooling modules includes three or more thermoelectric cooling modules.

5. The anti-condensation protective eyewear of claim 1, wherein the one or more thermoelectric cooling modules are configured to be electrically coupled to a mounting rail such that the one or more thermoelectric cooling modules receive power from the mounting rail.

6. The anti-condensation protective eyewear of claim 1, further comprising: a thermally conductive element coupled to the cooling surface and disposed within the interior chamber.

7. The anti-condensation protective eyewear of claim 1, wherein the sidewall structure is at least partially comprised of a flexible material.

8. The anti-condensation protective eyewear of claim 1, further comprising: a heat sink coupled to the heating surface of each of the one or more thermoelectric cooling modules.

9. The anti-condensation protective eyewear of claim 1, wherein the heating surface of each of the one or more thermoelectric cooling modules is coupled to the outer surface of the sidewall structure and is exterior to the interior chamber.

10. The anti-condensation protective eyewear of claim 1, wherein the one or more thermoelectric cooling modules are in electrical communication with a power source coupled to a helmet.

11. The anti-condensation protective eyewear of claim 1, wherein the lens is comprised of a ballistic grade material.

12. The anti-condensation protective eyewear of claim 1, wherein the lens includes one or more laser absorptive dyes, laser reflective coatings or a combination thereof.

13. The anti-condensation protective eyewear of claim 1, wherein the lens includes a hydrophobic coating.

14. The anti-condensation protective eyewear of claim 1, wherein the lens includes one or more variable light transmissive materials including at least one of a photochromic material, an electrochromic material, and liquid crystal technology.

15. The anti-condensation protective eyewear of claim 1 further comprising:
a display device configured to project an image to a wearer's eye.

16. The anti-condensation protective eyewear of claim 15, wherein the display device includes one or more waveguides

17. The anti-condensation protective eyewear of claim 1, further comprising:
at least one temperature sensor coupled to the inner surface of the sidewall structure, the inner surface of the lens or a combination of both, the at least one temperature sensor configured to generate a signal including an indication of at least one of a temperature inside the interior chamber and a temperature of the inner surface of the lens; and
a controller in communication with the at least one temperature sensor and coupled to the one or more thermoelectric cooling modules, the controller configured to selectively provide power to the one or more thermoelectric cooling modules in response receiving the signal from the at least one temperature sensor.

18. The anti-condensation protective eyewear of claim 17, wherein the controller is configured to provide power to the one or more thermoelectric cooling modules in response to the signal indicating that the temperature inside the interior chamber or the temperature of the inner surface of the lens is above a predetermined threshold.

19. The anti-condensation protective eyewear of claim 17, wherein the controller is configured to cease providing power to the one or more thermoelectric cooling modules in response to the signal indicating that the temperature inside the interior chamber or the temperature of the inner surface of the lens is below a predetermined lower threshold.

20. The anti-condensation protective eyewear of claim 1, wherein the sidewall structure is a flexible gasket coupled to the periphery of the lens to form a continuous seal between the lens and flexible gasket.

21-24. (canceled)

25. The anti-condensation protective eyewear of claim 1, wherein the one or more thermoelectric cooling modules extend through the sidewall structure.

26. The anti-condensation protective eyewear of claim 1, wherein the lens is a dual lens structure including an inner lens and an outer lens spaced from the inner lens, the space between the inner lens and outer lens defining a lens gap, and
wherein the heating surface of the one or more thermoelectric cooling modules is thermally connected to the lens gap.

27. The anti-condensation protective eyewear of claim 1, wherein heat generated by the heating surface of at least one of the one or more thermoelectric cooling modules is configured to be transferred to a location or device on or proximate a user wearing the anti-condensation protective eyewear.

28. An anti-condensation protective eyewear comprising:
a lens having an outer periphery, an inner surface, and an outer surface;
a sidewall structure coupled to the periphery of the lens, the sidewall structure having an inner surface and an outer surface, a top surface, a bottom surface, a first side surface, and a second side surface, the inner surface of the sidewall structure and the inner surface of the lens defining an interior chamber; and
four or more thermoelectric cooling modules coupled to the sidewall structure, each thermoelectric cooling module of the four or more thermoelectric cooling modules comprising:
a cooling surface disposed on a first side of the thermoelectric cooling module,
a heating surface disposed on a second side, opposite the first side, of the thermoelectric cooling module;
a thermally conductive element coupled to the cooling surface; and
a heat sink coupled to the heating surface,
wherein the four or more thermoelectric cooling modules include a first thermoelectric cooling module and a second thermoelectric cooling module each coupled to the top surface of the sidewall structure, a third thermoelectric cooling module coupled to the first side surface of the sidewall structure, and a fourth thermoelectric cooling module coupled to the second side surface of the sidewall structure,
wherein each thermoelectric cooling module of the four or more thermoelectric cooling modules extend through the sidewall structure such that the cooling surface of each of the four or more thermoelectric cooling modules is disposed within the interior chamber.
